(19) United States
(12) Patent Application Publication
(10) Pub. No.: US 2019/0167763 A1
(43) Pub. Date: Jun. 6, 2019

(54) TOPICAL PHARMACEUTICAL COMPOSITION FOR PROMOTING HAIR GROWTH AND/OR REDUCING HAIR LOSS

(71) Applicants: Habil F KHORAKIWALA, Aurangabad (IN); Vijay SHARMA, Aurangabad (IN); Zahabiya KHORAKIWALA, Aurangabad (IN)

(72) Inventors: Habil F KHORAKIWALA, Aurangabad (IN); Vijay SHARMA, Aurangabad (IN); Zahabiya KHORAKIWALA, Aurangabad (IN)

(21) Appl. No.: 16/318,354

(22) PCT Filed: Jul. 19, 2017
(86) PCT No.: PCT/IN2017/050296

§ 371 Date: Jan. 16, 2019

(30) Foreign Application Priority Data
Jul. 21, 2016 (IN) 201621025016
Jul. 21, 2016 (IN) 201621025017

(51) Int. Cl. A61K 38/18; A61K 9/107; A61K 9/12; A61Q 7/00; A61P 17/14

(52) U.S. Cl. CPC A61K 38/1858; A61K 9/107; A61K 38/178; A61Q 7/00; A61P 17/14; A61K 9/12

(57) ABSTRACT

The invention relates to a topical pharmaceutical composition for promoting hair growth and/or reducing hair loss in a human. In particular, the pharmaceutical composition comprises: (A) therapeutically effective amount of platelet component that includes a growth factor concentrate comprising platelet derived growth factor (PGDF), vascular endothelial growth factor (VEGF), fibroblast growth factor (FGF), and transforming growth factor-β (TGF-β); and (B) a permeability enhancing agent.

### TECHNICAL FIELD OF THE INVENTION

The invention relates to a topical pharmaceutical composition for promoting hair growth and reducing hair loss in a human. In particular, the composition comprises: (A) therapeutically effective amount of platelet component that includes a growth factor concentrate; and (B) a permeability enhancing agent.

### BACKGROUND OF THE INVENTION

Hair loss is a natural phenomenon. Hair growth follows a cycle which involves the birth and development of the follicle, a stationary phase, and a final phase during which the hair is expelled. This alternation between the phases of growth (the anagenic phase), regression (the catagenic phase), and the rest (the telegenic phase) is due to the specific secretion of the hair follicle which acts as a gland, and progressively produces a mass of keratin which it eliminates and replaces after a resting period.

The cycle begins with the development of the hair follicle that rises up from the dermis which contains large numbers of mesenchymatous cells, resulting in the formation of a dermal papilla. In the final stage (the anagenic phase) the cells surrounding the dermal papilla divide actively every 12 hours in order to produce cells which line up, grow longer, and begin to keratinize. This is hair growth. During the catagenic phase mitosis no longer occurs and the bulb detaches itself from the papilla and rises towards the surface. In the telegenic phase the hair is fully keratinized and is ready to be expelled. After three to four months, another mitotic cycle begins in the germination zone of the hair and another hair follicle is formed.

Hair loss or baldness or alopecia is a loss of hair from the head or body. Baldness can refer to general hair loss or androgenic alopecia (male pattern baldness), female pattern baldness involving gonadal hormones. Some types of baldness can be caused by alopecia areata, an autoimmune disorder. The extreme forms of alopecia areata are alopecia totalis, which involves the loss of all head hair, and alopecia universalis, which involves the loss of all hair from the head and the body.

In many individuals, alopecia causes embarrassment, psychological problems, including depression, and can affect one's self image and feelings of sexuality. Treatments for the various forms of hair loss have limited success. Three medications have evidence to support their use in male pattern hair loss: oral finasteride (PROPECIA®, Merck), oral dutasteride and topical minoxidil (ROGAINE®, Pfizer). These treatments require prolonged usage of the drug and if treatment is stopped any benefits gained will be lost and the hair thickness will regress to levels as if no treatment was undertaken. Reversible side effects associated with the use of these androgen inhibitors have been reported such as decreased libido, erectile dysfunction and dermatologic discomfort. Other modalities for treating hair loss include in-vivo hair transplantation whereby donor hair follicles from areas of the scalp that are insensitive to the effects of androgens are transplanted to areas experiencing irregular hair follicle cycling or areas with few hair follicles. Concerns about the efficacy and safety during the requisite long- term treatment of hair loss with the FDA-approved oral finasteride, topical minoxidil therapy prompted the use of newer therapies involving blood components isolated from the patient. This treatment, involves centrifuging a person's own blood until it contains a concentrated mixture of plasma cells and growth factors and then injecting the resulting substance directly into the injured tissue.

US Application Publication No. 2012/0087903A1 discloses hair growth agent having 5000 platelets per μL of solution. PCT Publication No. WO2014/047246 discloses a method of inducing or promoting hair growth on the scalp of a subject, comprising the steps of providing an extra cellular matrix composition. PCT Publication No. WO2014/027363 discloses a growth factor concentrate for treating hair loss. PCT Publication No. WO 2013/007308 discloses platelet-rich plasma (PRP) for epicutaneous use in a wide range of applications in the cosmetic and dermatology. U.S. Pat. No. 9,227,089 discloses infusible platelet-rich blood product wherein application involves traumatizing the skin to form a treatment area in which stem cells within the hair follicles are activated. PCT Publication No. WO2004/084825A2 discloses a wound healing composition derived from a low platelet concentration plasma preparation. European Patent No. EP2628484 discloses a pharmaceutical composition for oral administration comprising platelet-rich plasma. PCT Publication No. WO2009/049318 discloses freeze-dried plasma formats specifically designed for the trauma care field. PCT Publication No. WO2008/048228 discloses stabilized whole-cell plasma, which retains the integrity and overall stability of the proteins and other macromolecules of the plasma.

The therapies disclosed in prior art use certain components of blood and discard other components which are very important and useful for treatment of hair loss. Thus the therapies of prior-art require frequent and significantly more withdrawal of blood from patient to produce the desired composition to treat hair loss at the same time provide limited therapeutic efficacy.

There exists a need for the development of non-invasive, ideally topical which is easy to use, patient compliant compositions for promoting hair growth and reducing hair loss, which can be applied by the patient without the need of skilled healthcare professional.

### SUMMARY OF THE INVENTION

In one general aspect of the invention there is provided, a topical pharmaceutical composition for promoting hair growth and/or reducing hair loss in a human, said composition comprising (A) therapeutically effective amount of platelet component that includes a growth factor concentrate; and (B) a permeability enhancing agent.

In another aspect of the invention there is provided, a topical pharmaceutical composition for promoting hair growth and/or reducing hair loss in a human, said composition comprising (A) therapeutically effective amount of platelet component that includes a growth factor concentrate containing (i) not less than about 6,000 pg/mL of platelet derived growth factor (PGDF), and (ii) not less than about 600 pg/mL of vascular endothelial growth factor (VEGF); and (B) a permeability enhancing agent.

In another aspect, the growth factor concentrate further comprises (a) not less than about 20 pg/mL of fibroblast growth factor (FGF), and (b) not less than about 80,000 pg/mL of transforming growth factor-β (TGF-β).

In another general aspect of the invention there is provided, a topical pharmaceutical composition for promoting hair growth and/or reducing hair loss in a human, said composition comprising (A) therapeutically effective amount of platelet component that includes a growth factor concentrate comprising (i) about 6,000 to about 90,000 pg/mL of PGDF, and (ii) about 600 to about 4,000 pg/mL of VEGF, and (B) a permeability enhancing agent.

In another aspect, the growth factor concentrate further comprises (a) about 20 to about 60 pg/mL of FGF, and (b) about 80,000 to about 140,000 pg/mL of TGF-β. In another aspect, the growth factor concentrate further comprises not less than about 100 ng/mL of P-selectin. In another aspect, the growth factor concentrate further comprises about 100 to about 500 ng/mL of P-selectin. In another aspect, the growth factor concentrate comprises (i) about 10,000 to about 50,000 pg/mL of PGDF, and (ii) about 750 to about 3,000 pg/mL of VEGF. In another aspect, the growth factor concentrate comprises (i) about 15,000 to about 40,000 pg/mL of PGDF, and (ii) about 800 to about 2,500 pg/mL of VEGF. In another aspect, the growth factor concentrate comprises (i) about 20,000 to about 30,000 pg/mL of PGDF, and (ii) about 850 to about 1,500 pg/mL of VEGF. In another aspect, the growth factor concentrate comprises (a) about 25 to about 55 pg/mL of FGF, and (b) about 90,000 to about 120,000 pg/mL of TGF-β. In another aspect, the growth factor concentrate comprises (a) about 30 to about 50 pg/mL of FGF, and (b) about 95,000 to about 110,000 pg/mL of TGF-β. In another aspect, the growth factor concentrate comprises about 35 to about 45 pg/mL of FGF, and (b) about 100,000 to about 105,000 pg/mL of TGF-β. In another aspect, the growth factor concentrate comprising about 120 to about 400 ng/mL of P-selectin. In another aspect, the growth factor concentrate comprising about 130 to about 375 ng/mL of P-selectin. In another aspect, the growth factor concentrate comprising about 140 to about 350 ng/mL of P-selectin.

In another general aspect of the invention, there is provided, a method of treatment for promoting hair growth and/or reducing hair loss in a human by applying to the scalp of the human the topical pharmaceutical composition.

In another aspect of the invention there is provided, a pharmaceutical composition for promoting hair growth and/or reducing hair loss in a human.

In another general aspect, the topical pharmaceutical composition further comprises (i) a plasma component, and (ii) one or more pharmaceutically acceptable excipient.

In an aspect of the present invention, the platelet component and/or the plasma component described herein are of autologous origin. In another aspect, the platelet component and/or the plasma component described herein are of allogeneic origin.

In another general aspect of the invention, there is provided a topical pharmaceutical composition for promoting hair growth and/or reducing hair loss in a human, said composition comprising (A) therapeutically effective amount of platelet component that includes a growth factor concentrate comprising (i) about 20,000 to about 30,000 pg/mL of PGDF, (ii) about 850 to about 1,500 pg/mL of VEGF, (iii) about 35 to about 45 pg/mL of FGF, (iv) about 100,000 to about 105,000 pg/mL of TGF-β, (v) about 140 to about 350 ng/mL of P-selectin; and (B) a permeability enhancing agent.

In another aspect, the permeability enhancing agent comprises amino acids, surfactants, solvents, essential oils, lower alkanols, or a mixture thereof. In another aspect, the permeability enhancing agent comprises polyethylene glycol, dimethyl formamide or a mixture thereof. In another aspect, the composition is applied externally onto scalp of the human in need thereof. In another aspect, the composition is in the form of solution, suspension, emulsion, ointment, foam, paste, gel, cream, lotion, spray, powder, or soap. In another general aspect of the invention there is provided, a process of preparing the topical pharmaceutical composition, wherein the process comprises steps of:
a) extracting the plasma containing platelets from blood of a human by centrifugation of blood at about 2000-8000 rpm for about 2-15 minutes,
b) clotting the platelets in the plasma by adding calcium solution to obtain growth factor concentrate,
c) transferring the obtained growth factor concentrate devoid of any platelet debris in to another container; and
d) mixing of permeability enhancing agent and optionally, one or more pharmaceutically acceptable excipient with the growth factor concentrate of step c) to obtain the topical pharmaceutical composition.

In another general aspect of the invention, there is provided a method of treatment for promoting hair growth and/or reducing hair loss in a human by applying to the scalp of the human the topical pharmaceutical composition comprising (A) therapeutically effective amount of platelet component that includes a growth factor concentrate comprising (i) about 6,000 to about 90,000 pg/mL of PGDF, and (ii) about 600 to about 4,000 pg/mL of VEGF; and (B) a permeability enhancing agent.

In another general aspect of the invention, there is provided a topical pharmaceutical composition comprising (A) therapeutically effective amount of platelet component that includes a growth factor concentrate comprising (i) about 6,000 to about 90,000 pg/mL of PGDF, and (ii) about 600 to about 4,000 pg/mL of VEGF, and (B) a permeability enhancing agent, for promoting hair growth and/or reducing hair loss in a human.

### BRIEF DESCRIPTION OF THE DRAWINGS

FIGS. 1, 2 and 3 show the trichoscopy analysis images comparison of human subjects before use of topical composition as baseline and after use of the topical composition for 6 months.

FIGS. 4, 5 and 6 show the gross photographic comparison of human subjects before use of topical composition as baseline and after use of the topical composition for 6 months.

### DETAILED DESCRIPTION OF THE INVENTION

While the invention has been described in term of its specific embodiments, certain modification and equivalents will be apparent to those skilled in the art and are intended to be included within the scope of the invention.

The present invention provides a topical pharmaceutical composition for promoting hair growth and/or reducing hair loss in a human, said composition comprising (A) therapeutically effective amount of platelet component that includes a growth factor concentrate; and (B) a permeability enhancing agent.

Applicants have surprisingly found that the beneficial use of a platelet component that includes growth factor concentrate along with plasma component in promoting the hair growth. The compositions of the present invention are easy to use, stable and are highly effective in promoting the growth of the hair.

The term “topical” as employed herein relates to the external application of the invention at the site of hair loss, reduced hair growth or baldness. Accordingly, such topical compositions useful in the methods of the invention include those pharmaceutical forms in which the composition is applied externally by direct contact with the skin surface or scalp to be treated. The term “platelet component” referred herein contains, inter alia, growth factor concentrate. The growth factor concentrate includes various growth factors such as platelet derived growth factor (PDGF), fibroblast growth factor (FGF), vascular endothelial growth factors (VEGF) and transforming growth factor-β (TGF-β) and P-selectin. The platelet component may be autologous (i.e., of the same subject or patient) or allogeneic (i.e., of a different subject or patient, but of the same species).

The term “plasma component” refers to plasma comprising proteins such as fibrinogen, albumin and globulin. The plasma component may be autologous (i.e., of the same subject or patient) or allogeneic (i.e., of a different subject or patient, but of the same species).

As used herein, the term “treatment” refers to a beneficial or desired clinical result including, but not limited to, alleviation or amelioration of one or more symptoms of the alopecia or hair loss; promoting hair growth, diminishing the extent of the alopecia.

In one embodiment of the invention there is provided, a topical pharmaceutical composition for promoting hair growth and/or reducing hair loss in a human, said composition comprising (A) therapeutically effective amount of platelet component that includes a growth factor concentrate containing (i) not less than about 6,000 pg/mL of PGDF, and (ii) not less than about 600 pg/mL of VEGF; and (B) a permeability enhancing agent.

In one embodiment of the invention there is provided, a topical pharmaceutical composition for promoting hair growth and/or reducing hair loss in a human, said composition comprising (A) therapeutically effective amount of platelet component that includes a growth factor concentrate containing (i) not less than about 6,000 pg/mL of PGDF, and (ii) not less than about 600 pg/mL of VEGF; and (B) a permeability enhancing agent, and wherein the topical pharmaceutical composition further comprises (a) a plasma component, and (b) one or more pharmaceutically acceptable excipient.

In another embodiment, the growth factor concentrate further comprises (a) not less than about 20 pg/mL of FGF, and (b) not less than about 80,000 pg/mL of TGF-β.

In one embodiment of the invention, there is provided a topical pharmaceutical composition for promoting hair growth and/or reducing hair loss in a human, said composition comprising (A) therapeutically effective amount of platelet component that includes a growth factor concentrate comprising (i) about 6,000 to about 90,000 pg/mL of PGDF, and (ii) about 600 to about 4,000 pg/mL of VEGF; and (B) a permeability enhancing agent.

In one embodiment of the invention, there is provided a topical pharmaceutical composition for promoting hair growth and/or reducing hair loss in a human, said composition comprising (A) therapeutically effective amount of platelet component that includes a growth factor concentrate comprising (i) about 6,000 to about 90,000 pg/mL of PGDF, and (ii) about 600 to about 4,000 pg/mL of VEGF; and (B) a permeability enhancing agent; and wherein the topical pharmaceutical composition further comprises (a) a plasma component, and (b) one or more pharmaceutically acceptable excipient.

In an aspect of the present invention, the platelet component and/or the plasma component described herein are of autologous origin. In another aspect, the platelet component and/or the plasma component described herein are of allogeneic origin.

In another embodiment, the growth factor concentrate comprises (i) about 10,000 to about 50,000 pg/mL of PGDF, and (ii) about 750 to about 3,000 pg/mL of VEGF.

In another embodiment, the growth factor concentrate comprises (i) about 15,000 to about 40,000 pg/mL of PGDF, and (ii) about 800 to about 2,500 pg/mL of VEGF.

In another embodiment, the growth factor concentrate comprises (i) about 20,000 to about 30,000 pg/mL of PGDF, and (ii) about 850 to about 1,500 pg/mL of VEGF.

In yet another embodiment, the growth factor concentrate further comprises (a) about 20 to about 60 pg/mL of FGF, and (b) about 80,000 to about 140,000 pg/mL of TGF-β.

In another embodiment, the growth factor concentrate comprises (a) about 25 to about 55 pg/mL of FGF, and (b) about 90,000 to about 120,000 pg/mL of TGF-β.

In another embodiment, the growth factor concentrate comprises (a) about 30 to about 50 pg/mL of FGF, and (b) about 95,000 to about 110,000 pg/mL of TGF-β.

In another embodiment, the growth factor concentrate comprises about 35 to about 45 pg/mL of FGF, and (b) about 100,000 to about 105,000 pg/mL of TGF-β.

In another embodiment, the growth factor concentrate further comprises not less than about 100 ng/mL of P-selectin.

In still another embodiment, the growth factor concentrate further comprises about 100 to about 500 ng/mL of P-selectin.

In another embodiment, the growth factor concentrate comprises about 120 to about 400 ng/mL of P-selectin.

In another embodiment, the growth factor concentrate comprises about 130 to about 375 ng/mL of P-selectin.

In another embodiment, the growth factor concentrate comprises about 140 to about 350 ng/mL of P-selectin.

In further embodiment of the invention, there is provided a topical pharmaceutical composition for promoting hair growth and/or reducing hair loss in a human, said composition comprising (A) therapeutically effective amount of platelet component that includes a growth factor concentrate comprising (i) about 6,000 to about 90,000 pg/mL of PGDF, (ii) about 600 to about 4,000 pg/mL of VEGF, (iii) about 20 to about 60 pg/mL of FGF, (iv) about 80,000 to about 140,000 pg/mL of TGF-I3, (v) about 100 to about 500 ng/mL of P-selectin; and (B) a permeability enhancing agent.

In further embodiment of the invention, there is provided a topical pharmaceutical composition for promoting hair growth and/or reducing hair loss in a human, said composition comprising (A) therapeutically effective amount of platelet component that includes a growth factor concentrate comprising (i) about 10,000 to about 50,000 pg/mL of PGDF, (ii) about 750 to about 3,000 pg/mL of VEGF, (iii) about 25 to about 55 pg/mL of FGF, (iv) about 90,000 to about 120,000 pg/mL of TGF-I3, (v) about 120 to about 400 ng/mL of P-selectin; and (B) a permeability enhancing agent.

In further embodiment of the invention, there is provided a topical pharmaceutical composition for promoting hair growth and/or reducing hair loss in a human, said composition comprising (A) therapeutically effective amount of platelet component that includes a growth factor concentrate comprising (i) about 15,000 to about 40,000 pg/mL of PGDF, (ii) about 800 to about 2,500 pg/mL of VEGF, (iii) about 30 to about 40 pg/mL of FGF, (iv) about 95,000 to about 110,000 pg/mL of TGF-I3, (v) about 130 to about 375 ng/mL of P-selectin; and (B) a permeability enhancing agent.

In further embodiment of the invention, there is provided a topical pharmaceutical composition for promoting hair growth and/or reducing hair loss in a human, said composition comprising (A) therapeutically effective amount of platelet component that includes a growth factor concentrate comprising (i) about 20,000 to about 30,000 pg/mL of PGDF, (ii) about 850 to about 1,500 pg/mL of VEGF, (iii) about 35 to about 45 pg/mL of FGF, (iv) about 100,000 to about 105,000 pg/mL of TGF-I3, (v) about 140 to about 350 ng/mL of P-selectin; and (B) a permeability enhancing agent.

In further embodiment of the invention, there is provided a topical pharmaceutical composition for promoting hair growth and/or reducing hair loss in a human, said composition comprising (A) therapeutically effective amount of platelet component that includes a growth factor concentrate comprising (i) about 20,000 to about 30,000 pg/mL of PGDF, (ii) about 850 to about 1,500 pg/mL of VEGF, (iii) about 35 to about 45 pg/mL of FGF, (iv) about 100,000 to about 105,000 pg/mL of TGF-I3, (v) about 140 to about 350 ng/mL of P-selectin; and (B) a permeability enhancing agent; and wherein the topical pharmaceutical composition further comprises (a) a plasma component, and (b) one or more pharmaceutically acceptable excipient.

Following is a table comprising different ranges of growth factor concentrate of present invention in pg/mL:

| Growth Factor | |
|---|---|
| Concentrate | Ranges in pg/mL |
| PGDF | 6,000 to 90,000; or 10,000 to 90,000; or 15,000 to |
| | 90,000; or 20,000 to 90,000 |
| VEGF | 600 to 4,000; or 750 to 4,000; or 800 to 4,000; or 850 to |
| | 4,000 |
| FGF | 20 to 60; or 25 to 60; or 30 to 60; or 35 to 60 |
| TGF-β | 80,000 to 140,000; or 90,000 to 140,000; or 95,000 to |
| | 140,000; or 100,000 to 140,000 |

In another embodiment, the topical pharmaceutical composition is applied externally onto scalp of the human in need thereof. In another embodiment, the topical pharmaceutical composition is applied externally onto scalp of the human in need thereof daily for male and alternate days for female.

A “permeability enhancing agent” as used herein includes natural permeability enhancers, synthetic permeability enhancers, and/or combination thereof and is selected from amino acids, surfactants, solvents, essential oils and lower alkanol, propylene glycol or a mixture of polyethylene glycols. The amount of permeability enhancing agent ranges from about 1% to about 50% by weight of composition. The amount of permeability enhancing agent ranges from about 5% to 30% by weight of composition. Alternatively, in an embodiment the amount of permeability enhancing agent is about 2%, or 3%, or 4%, or 5%, or 6%, or 10%, or 15%, or 20%, or 30%. Each amount constitutes an alternate embodiment of the present invention.

In an embodiment, the amino acids selected from the group comprising glycine, argininine, histidine, tuarine, glutamine and methionine. Each of these amino acids constitutes an alternate embodiment of the invention. In an embodiment, the glycine concentration can range from about 1% to about 50% or about 1% to about 30% or about 1% to about 20% or about 1% to about 10%. Alternatively, the glycine concentration can range from about 3%, or 4%, or 5%, or 6%, or 10%, or 15%, or 20%, or 30% by weight of composition. Each of these ranges constitutes an alternate embodiment of the invention.

In each of the embodiment, the amino acid glycine can be readily replaced by other amino acids like arginine, histidine, methionine and like in mentioned concentration ranges of embodiments and would constitute an alternate embodiment of the invention. For example; arginine present in an amount of about 6% or 10% by weight and histidine present in an amount of about 9% or 15% by weight of composition.

A “surfactants” as used herein can be selected form the group comprising of one or more of sodium bis(2-ethylhexyl)sulfosuccinate, sodium bis(tridecyl)sulfosuccinate, bis(dialkyl)sulfosuccinate salts, copolymers of polydimethylsiloxane and polyethylene/polypropylene-oxide, polyoxypropylene (12) dimethicone, cetyl PEG/PPG-10/1 dimethicone, hexyl laurate and polyglyceryl-4-isostearate, PEG-10 dimethicone, sorbitan monolaurate, sorbitan monooleate, polyoxyethylene (20) sorbitan monooleate (Polysorbate 80), polyethoxylated castor oil, polyoxyethylenesorbitan trioleate, polyoxyethylene octyl phenyl ether, polyoxyethylene 20 cetyl ether, polyethylene glycol tert-octylphenyl ether, sodium di(2-ethylhexyl)phosphate, sodium di(oleyl)phosphate, sodium di(tridecyl)phosphate, sodium dodecylbenzenesulfonate, sodium 3-dodecylaminopropanesulfonate, sodium 3-dodecylaminopropionate, sodium N-2-hydroxydodecyl-N-methyltaurate, lecithin, sucrose fatty acid esters, 2-ethylhexylglycerin, caprylyl glycol, long chain hydrophobic vicinal diols of monoalkyl glycols, monoalkyl glycerols, or monoacyl glycerols, polyoxyl castor oil derivatives, polyethylene glycol hydrogenated castor oil, potassium oleate, sodium oleate, cetylpyridynium chloride, alkyltrimethylammonium bromides, benzalkonium chloride, didodecyldimethylammonium bromide, trioctylmethylammonium bromide, cetyltrimethylammonium bromide, cetyldimethylethylammonium bromide, and combinations thereof. The preferred surfactants are polysorbate 80, sodium oleate, lecithin, sucrose fatty acid esters, and polyoxyl castor oil derivatives. Each constitutes an alternate embodiment of the invention. Most preferred is polysorbate 80.

The amount of surfactants (e.g., polysorbate 80) is ranges from about 1% to about 50% by weight of composition. The preferred amount is about 2%, or 4%, or 6%, or 10%, or 15%, or 20%, or 30% by weight of composition.

The essential oil as used herein are selected from eucalyptus oil, chenopodium oil, tea tree oil, cumin oil, rose oil and aloe vera oil. Each of these oils constitutes an independent embodiment, of the invention. The preferred oils are selected from eucalyptus oil and chenopodium oil. The concentration of oil ranges from about 1% to about 50% by weight of composition. The preferred amount is about 2%, or 4%, or 6%, or 10%, or 15%, or 20%, or 30% by weight of composition.

The lower alkanol, polyethylene glycol or mixture of polyethylene glycol is present in concentration of about 1% to 50% by weight of composition. The preferred amount is about 2%, or 4%, or 6%, or 10%, or 15%, or 20%, or 30% by weight of composition. Most preferred is polyethylene glycol.

The “solvent” is a compound, such as one or more of water, ethanol, menthol, thymol, benzyl alcohol, isopropyl alcohol, propylene glycol, dimethylformamide (DMF), methylated spirit, phenol, ethyl oleate, glycerol, levomenol, monoethanolamine oleate, myristyl alcohol, octyldodecanol, coconut oil or silicone oil and combination thereof. Preferred solvent is DMF in an amount of 5-15% by weight of composition.

In another embodiment, the permeability enhancing agent comprises polyethylene glycol, dimethyl formamide or a mixture thereof.

In another embodiment the permeability enhancing agent comprises polyethylene glycol in an amount of 10-20% by weight of composition, dimethyl formamide in an amount of 5-10% by weight of composition or a mixture thereof.

In an embodiment of the invention the topical pharmaceutical composition comprises one or more pharmaceutically acceptable excipients selected from thickening agents, buffers, antioxidants, stabilizers and solvents.

A “thickening agent” as used herein include, but not limited to one or more of anionic cellulose materials, such as sodium carboxy methyl cellulose; anionic polymers such as carboxy vinyl polymers; nonionic cellulose materials, such a methyl cellulose and hydroxy propyl methyl cellulose; cationic cellulose materials, such as Polymer JR 400; cationic gum materials, such as Jaguar C₁₃ S; other gum materials such as gum acacia, gum tragacanth, locust bean gum, guar gum and carrageenan; proteins, such as albumin and protein hydrolysates; and clay materials, such as bentonite, hectorite, magnesium aluminium silicate, sodium magnesium silicate and combination thereof. Preferred thickening agent is sodium carboxy methyl cellulose.

The concentration of thickening agent (e.g., sodium carboxy methyl cellulose) is ranges from about 0.2% to about 5% by weight of composition. Alternatively, about 0.2%, or about 0.4%, or about 0.6%, or about 0.8%, or about 1%, or about 1.2%, or about 1.6%, or about 1.8%, or about 2%, or about 3%, or about 4%, or about 5% by weight composition. Each of this concentration constitutes an alternate embodiment of the invention. Preferred concentration is about 1.2%, or about 1.6%, or about 1.8%, or about 2% by weight of composition.

The “preservatives” as used herein include, but are not limited to one or more of ethanol, benzoic acid, sodium benzoate, sorbic acid, potassium sorbate, sodium propionate and the methyl, ethyl, propyl and butyl esters of p-hydroxybenzoic acid 2-bromo-2-nitropropane-1,3-diol, phenoxyethanol, dibromodicyanobutane, formalin, triclosan and combination thereof. The preferred preservative is sodium benzoate.

The concentration of preservative (e.g., sodium benzoate) is ranges from 0.1% to 2% by weight of composition. Alternatively, about 0.1%, or about 0.15%, or about 0.2%, or about 0.25%, or about 0.3%, or about 0.35%, or about 0.4%, or about 0.45%, or about 0.5%, or about 0.55%, or about 0.6%, or about 0.65%, or about 0.7%, or about 0.75%, or about 0.8%, or about 0.85%, or about 0.9%, or about 0.95%, or about 1%, or about 2% by weight of composition. Preferred concentration is about 0.3%, or about 0.45%, or about 0.5%, or about 0.65%, or about 0.75% by weight of composition.

The “buffering agents” as used herein include, but are not limited to one or more of citric acid, citric acid monohydrate, boric acid, and phosporic acid, sodium citrate, sodium citrate dihydrate, monopotassium phosphate, disodium phosphate and combination thereof. Preferred buffering agent is citric acid monohydrate.

An suitable “antioxidants” as used herein can be selected form the group comprising of one or more of acetyl cysteine, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, tris(nonylphenyl)phosphate, and combination thereof.

In another embodiment, the composition is in the form of solution, suspension, emulsion, ointment, foam, paste, gel, cream, lotion, spray, powder, or soap.

In another embodiment of the invention there is provided, a process of preparing the topical pharmaceutical composition, wherein the process comprises steps of:
a) extracting the plasma containing platelets from blood of a human by centrifugation of blood at about 2000-8000 rpm for about 2-15 minutes,
b) clotting the platelets in the plasma by adding calcium solution to obtain growth factor concentrate,
c) transferring the obtained growth factor concentrate devoid of any platelet debris in to another container; and
d) mixing of permeability enhancing agent and optionally, one or more pharmaceutically acceptable excipient with the growth factor concentrate of step c) to obtain the topical pharmaceutical composition.

In another embodiment of the invention there is provided, a process of preparing the topical pharmaceutical composition, wherein the process comprises steps of:
a) extracting the plasma containing platelets from blood of a human by centrifugation of blood at about 3000-4000 rpm for about 5 minutes,
b) clotting the platelets in the plasma by adding calcium solution to obtain growth factor concentrate,
c) transferring the obtained growth factor concentrate devoid of any platelet debris in to another container; and
d) mixing of permeability enhancing agent and optionally, one or more pharmaceutically acceptable excipient with the growth factor concentrate of step c) to obtain the topical pharmaceutical composition.

In another embodiment of the invention, final volume of topical pharmaceutical composition is in amount of 5-15 mL, preferably 6-12 mL or 8-10 mL. In another embodiment of the invention, dosing amount for application of topical pharmaceutical composition is 0.5 mL to 1 mL each time.

In another embodiment of the invention, there is provided a method of treatment for promoting hair growth and/or reducing hair loss in a human by applying to the scalp of the human the topical pharmaceutical composition comprising (A) therapeutically effective amount of platelet component that includes a growth factor concentrate comprising (i) about 6,000 to about 90,000 pg/mL of PGDF, and (ii) about 600 to about 4,000 pg/mL of VEGF, and (B) a permeability enhancing agent.

In another embodiment of the invention, there is provided a topical pharmaceutical composition comprising (A) therapeutically effective amount of platelet component that includes a growth factor concentrate comprising (i) about 6,000 to about 90,000 pg/mL of PGDF, and (ii) about 600 to about 4,000 pg/mL of VEGF, and (B) a permeability enhancing agent, for promoting hair growth and/or reducing hair loss in a human.

In another embodiment of the invention, there is provided an applicator, for storing the topical pharmaceutical composition, wherein the applicator comprises:
a) a reservoir for holding the topical pharmaceutical composition
b) a connector channel in communication with reservoir, and
c) a pad serving to seal the reservoir at a breakable connection, and wherein breaking of said breakable connection allows passage of the composition from the reservoir into the pad.

### EXAMPLES

The examples supporting the invention are described as below. The following example is given by the way of illustration of the present invention and therefore should not be construed to limit the scope of the present invention.

### Example 1

A Topical pharmaceutical composition of growth factor concentrate

TABLE 1

| Sr. | | |
|---|---|---|
| No. | Ingredients | Quantity/mL |
| A | Platelet component that includes | |
| | growth factor concentrate | |
| 1. | Platelet derived growth factor | 6,000 to 90,000 pg/mL |
| | (PGDF) | |
| 2. | Vascular endothelial growth factor | 600 to 4,000 pg/mL |
| | (VEGF) | |
| 3. | Fibroblast growth factor (FGF) | 20 to 60 pg/mL |
| 4. | Transforming growth factor-β | 80,000 to 140,000 pg/mL |
| | (TGF-β) | |
| 5. | P-selectin | 100 to 500 ng/mL |
| B | Plasma Component | q.s. to 10 mL |
| C | i) Polyethelene glycol | 1 to 2 mL |
| | ii) Dimethylformamide | 0.5 to 1.5 mL |
| | Total | 10 mL |

### Example 1A

A Topical pharmaceutical composition of growth factor concentrate

TABLE 2

| Sr. | | |
|---|---|---|
| No. | Ingredients | Quantity/mL |
| A | Platelet component that includes | |
| | growth factor concentrate | |
| 1. | Platelet derived growth factor | 20,000 to 30,000 pg/mL |
| | (PGDF) | |
| 2. | Vascular endothelial growth factor | 850 to 1,500 pg/mL |
| | (VEGF) | |
| 3. | Fibroblast growth factor (FGF) | 35 to 45 pg/mL |
| 4. | Transforming growth factor-β | 100,000 to 105,000 pg/mL |
| | (TGF-β) | |
| 5. | P-selectin | 140 to 350 ng/mL |
| B | Plasma Component | q.s. to 10 mL |
| | Total | 10 mL |

Preparation procedure:

The topical pharmaceutical composition of Example 1 and Example 1A was prepared by following process
a) Blood was collected from a human subject, from which plasma containing platelets was extracted by centrifugation at about 4000 rpm for about 5 minutes,
b) platelets in the plasma obtained in step (a) were subjected for clotting by adding calcium solution to obtain growth factor concentrate,
c) growth factor concentrate obtained in step (b) along with plasma component (devoid of any cellular debris) were transferred in to another container, and
d) mixing of permeability enhancing agent and optionally one or more pharmaceutically acceptable excipient with growth factor concentrate to obtain the topical pharmaceutical composition.

### Example 2

Comparison of commercially available growth factor concentrate compositions with composition of Example 1

Six different commercially available growth factor concentrate containing composition (C1, C2, C3, C4, C5 and C6) were compared with the topical pharmaceutical composition of Example 1 for concentration of various growth factors.

| Sr. | Commercial | |
|---|---|---|
| No. | Compositions | Brand Name of commercial compositions |
| 1 | C1 | RegenKit BCT by Regan Lab USA |
| 2 | C2 | Plasmolifting by Italtrade & CO |
| 3 | C3 | Rejsol PRP Kit by Rejsol |
| 4 | C4 | BD Vacutainer SST II Advance tube |
| 5 | C5 | DrPRP, by Dr. PRP USA LLC |
| 6 | C6 | YCellBio PRP by Ycellbio Medical Co., Ltd. |

Applicants of the present inventions have surprisingly found that composition of Example 1 has very high concentration of growth factors as compared to other commercially available compositions as shown in Table 3.

TABLE 3

| | | (Quantity/mL) | | | | | | |
|---|---|---|---|---|---|---|---|---|
| Sr. | | | Commercially available compositions | | | | | |
| No. | Ingredients | Example 1 | C1 | C2 | C3 | C4 | C5 | C6 |
| 1. | Platelet derived | 6,000 to 90,000 | 500 to 2150 | 170 to 2600 | 500 to 2700 | 1,500 to 5,000 | 10 to 100 | 35 to 400 |
| | growth factor | | | | | | | |
| | (PGDF) pg/mL | | | | | | | |
| 2. | Vascular | 600 to 4,000 | 9 to 35 | 10 to 80 | 20 to 250 | 40 to 500 | 5 to 25 | 10 to 25 |
| | endothelial | | | | | | | |
| | growth factor | | | | | | | |
| | (VEGF) pg/mL | | | | | | | |
| 3. | Fibroblast growth | 20 to 60 | 8 to 10 | 8 to 10 | 8 to 10 | 8 to 10 | 8 to 10 | 0.5 to 2 |
| | factor (FGF) | | | | | | | |
| | pg/mL | | | | | | | |
| 4. | Transforming | 80,000 to 140,000 | 11,000 to 35,500 | 3,600 to 47,000 | 9,500 to 21,000 | 35,000 to 75,000 | 1,000 to 5,000 | 200 to 1,500 |
| | growth factor-β | | | | | | | |
| | (TGF-β) pg/mL | | | | | | | |
| 5. | P-selectin ng/mL | 100 to 500 | 25 to 50 | 20 to 55 | 20 to 50 | 35 to 70 | 15 to 40 | 15 to 40 |

### Example 3

Treatment of human subject with the topical pharmaceutical composition of Example 1

The topical pharmaceutical composition of Example 1 was subjected for study to characterise its efficacy in promoting hair growth and reducing hair loss. Trichoscopy analysis was performed at baseline and followed by a time interval of 2 months, 3 months, 4 months, 5 months and 6 months to determine total percentage increase in hair count and hair thickness of total 73 study subjects.

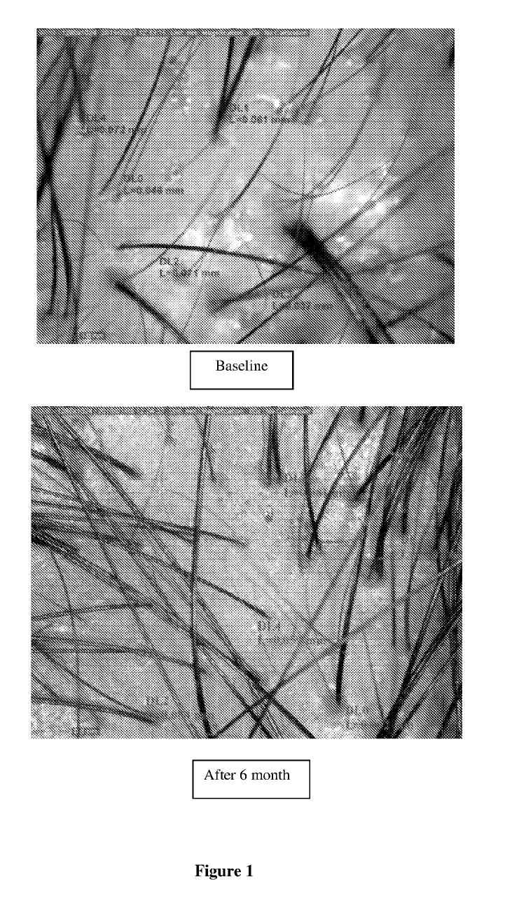

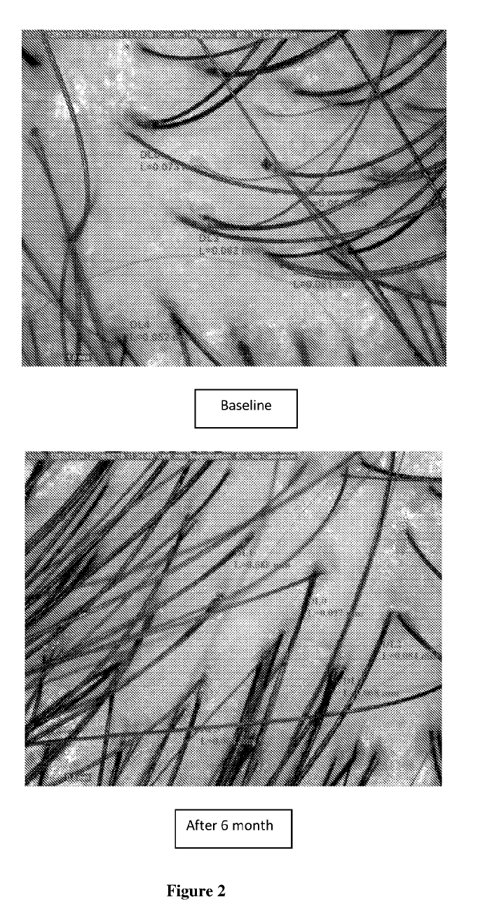

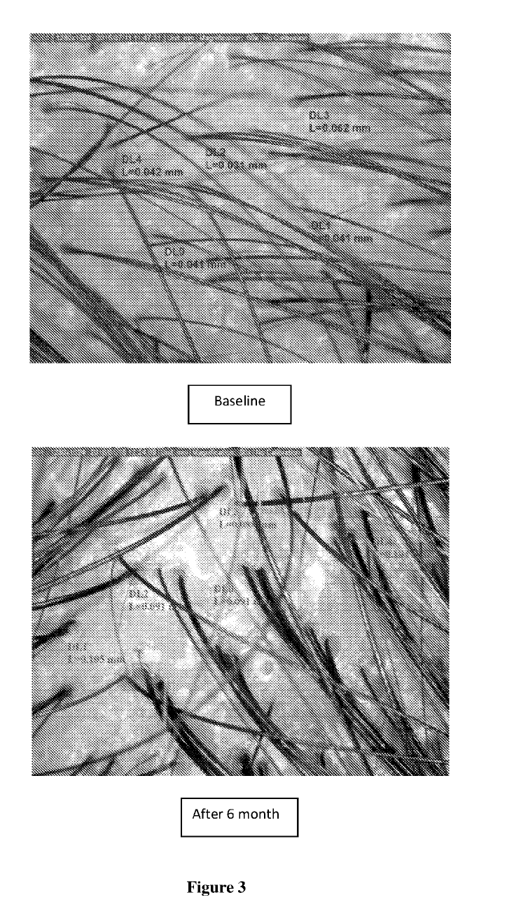

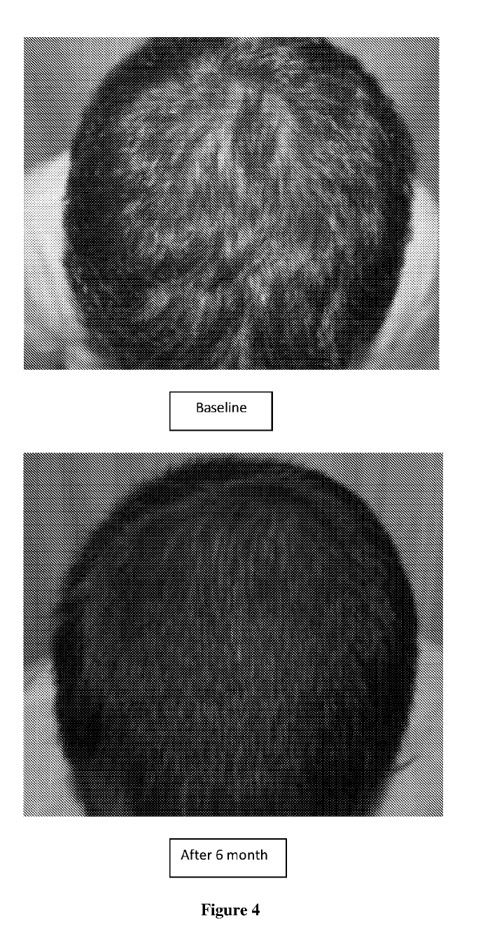

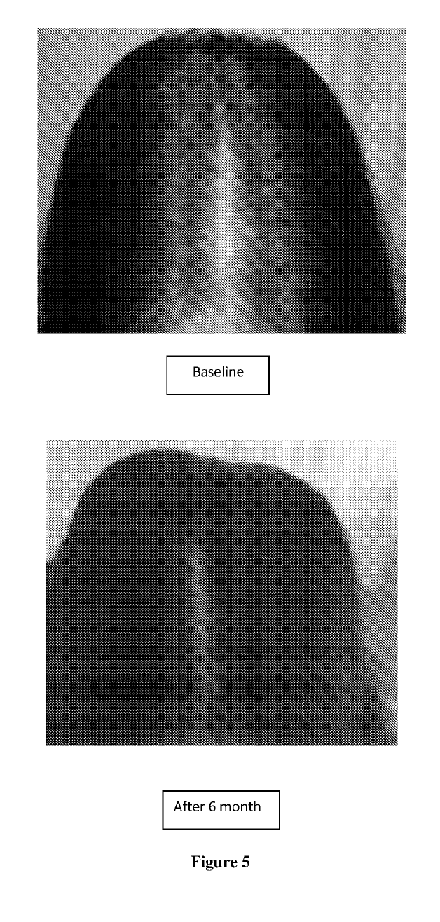

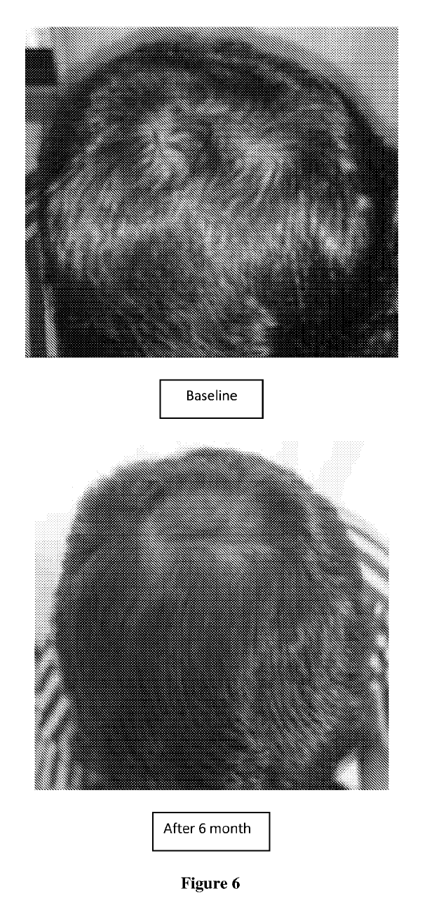

Result given in Table 4 and FIG. 1, FIG. 2, and FIG. 3 show that the percentage increase in hair count and hair thickness continued to improve from baseline to 6 months with the usage of topical composition of Example 1. FIG. 4, FIG. 5 and FIG. 6 demonstrate that there is a significant increase in total hair count and hair thickness.

TABLE 4

| Parameter | 2 months | 3 months | 4 months | 5 months | 6 months |
|---|---|---|---|---|---|
| % Increase | 10.37 | 28.12 | 57.53 | 66.79 | 78.14 |
| in hair | | | | | |
| count | | | | | |
| % Increase | 8.79 | 68.79 | 74.4 | 85.37 | 96.13 |
| in hair | | | | | |
| thickness | | | | | |

As it is evident from the above result, the topical pharmaceutical composition of the invention serves to provide consistent positive results in promoting hair growth and reducing hair loss in human. Total 78.14% increase in hair count and 96.13% increase in hair thickness was observed after 6 month usage of the topical composition.

### Example 4

Topical Solution of growth factor concentrate

TABLE 5

| Sr. No. | Ingredients | Quantity/mL |
|---|---|---|
| A | Platelet component that includes | |
| | growth factor concentrate | |
| 1. | Platelet derived growth factor | 20,000 to 30,000 pg/mL |
| | (PGDF) | |
| 2. | Vascular endothelial growth factor | 850 to 1,500 pg/mL |
| | (VEGF) | |
| 3. | Fibroblast growth factor (FGF) | 35 to 45 pg/mL |
| 4. | Transforming growth factor-β | 100,000 to 105,000 pg/mL |
| | (TGF-β) | |
| 5. | P-selectin | 140 to 350 ng/mL |
| B | Plasma Component | q.s. to 10 mL |
| C | i) Polythelene glycol | 1 to 2 mL |
| | ii) Dimethylformamide | 0.5 to 1.5 mL |
| | iii) Sodium carboxymethyl | 0.1 to 0.5 mg |
| | Cellulose | |
| | iv) Sodium Benzoate | 0.5 to 1 mg |
| | Total | 10 mL |

Preparation procedure: The topical solution of Example 4 was prepared by process mentioned in Example 1.

1. A topical pharmaceutical composition for promoting hair growth and/or reducing hair loss in a human, said composition comprising (A) a platelet component that includes a growth factor concentrate containing (i) about 6,000 pg/mL to about 90,000 pg/mL of platelet derived growth factor (PDGF), and (ii) about 600 pg/mL to about 4,000 pg/mL of vascular endothelial growth factor (VEGF).

2. The pharmaceutical composition according to claim 1, wherein the growth factor concentrate further comprises (a) about 20 pg/mL to about 60 pg/mL of fibroblast growth factor (FGF), and (b) about 80,000 pg/mL to about 140,000 pg/mL of transforming growth factor-β (TGF-β).

3. The topical pharmaceutical composition according to claim 1, wherein the composition further comprises a permeability enhancing agent.

4. The pharmaceutical composition according to claim 1, wherein the growth factor concentrate further comprises (a) about 30 to about 40 pg/mL of fibroblast growth factor (FGF), and (b) about 95,000 to about 110,000 pg/mL of transforming growth factor-β (TGF-β).

5. The pharmaceutical composition according to claim 1, wherein the growth factor concentrate further comprises about 100 ng/mL to about 500 ng/mL of P-selectin.

6. The pharmaceutical composition according to claim 1, wherein the growth factor concentrate further comprises about 120 to about 400 ng/mL of P-selectin.

7. The pharmaceutical composition according to claim 3, wherein the permeability enhancing agent comprises amino acids, surfactants, solvents, essential oils, lower alkanols, or a mixture thereof.

8. The pharmaceutical composition according to claim 1, wherein the platelet component is of autologous or allogeneic origin.

9. The pharmaceutical composition according to claim 1, wherein the composition is applied externally onto scalp of the human in need thereof.

10. The pharmaceutical composition according to claim 1, wherein the composition is in the form of a solution, suspension, emulsion, ointment, foam, paste, gel, cream, lotion, spray, powder, or soap.

11. A process of preparing the topical pharmaceutical composition according to claim 3, wherein the process comprises steps of:
a) extracting the plasma containing platelets from blood of a human by centrifugation of blood at about 2000-8000 rpm for about 2-15 minutes;
b) clotting the platelets in the plasma by adding calcium solution to obtain growth factor concentrate;
c) transferring the obtained growth factor concentrate devoid of any platelet debris in to another container; and
d) mixing of permeability enhancing agent and optionally, one or more pharmaceutically acceptable excipient with the growth factor concentrate of step c) to obtain the topical pharmaceutical composition.

12. A method of treatment for promoting hair growth and/or reducing hair loss in a human by applying to the scalp of the human the topical pharmaceutical composition according to claim 1.

13. The pharmaceutical composition according to claim 7, wherein the permeability enhancing agent comprises polyethylene glycol, dimethyl formamide or a mixture thereof.

14. A topical pharmaceutical composition for promoting hair growth and/or reducing hair loss in a human said composition comprising (A) a platelet component that includes a growth factor concentrate containing (i) about 6,000 pg/mL to about 90,000 pg/mL of platelet derived growth factor (PDGF), (ii) about 600 pg/mL to about 4,000 pg/mL of vascular endothelial growth factor (VEGF), (iii) about 20 pg/mL to about 60 pg/mL of fibroblast growth factor (FGF), (iv) about 80,000 pg/mL to about 140,000 pg/mL of transforming growth factor-β (TGF-β), and (v) about 100 ng/mL to about 500 ng/mL of P-selectin.

15. The pharmaceutical composition of claim 14, wherein the composition further comprises permeability enhancing agent comprising about (i) 1 mL to about 2 mL of polyethelene glycol and (ii) about 0.5 mL to about 1.5 mL of dimethylformamide.
